(19) United States
(12) Patent Application Publication
(10) Pub. No.: US 2026/0237508 A1
(43) Pub. Date: Aug. 13, 2026

(54) AI-BASED STRUCTURE CONTOURING USING CROSS-ATTENTION SEGMENTATION

(71) Applicant: Siemens Healthineers International AG, Steinhausen (CH)

(72) Inventors: Zhoubing XU, Princeton, NJ (US); Sasa GRBIC, Plainsboro, NJ (US)

(73) Assignee: Siemens Healthineers International AG, Steinhausen (CH)

(21) Appl. No.: 19/053,039

(22) Filed: Feb. 13, 2025

(51) Int. Cl. G16H 50/20; G16H 20/40; G16H 30/40

(52) U.S. Cl. CPC G16H 50/20; G16H 20/40; G16H 30/40

(57) ABSTRACT

A method can include generating a training dataset at least one query medical image depicting one or more structures within an anatomical region, and a plurality of support medical images depicting the one or more structures within the anatomical region. The method can include generating a feature embedding for the at least one query medical image and the plurality of the support medical images. The method can include training a machine learning model using the training dataset and the feature embedding, such that the machine learning model is configured to a contouring attribute for a first structure within a new medical image in accordance with a spatial correspondence to a second structure within the new medical image. The method can include transmitting the contouring attribute to a radiotherapy computer model configured to generate a radiotherapy treatment plan for treatment of the first structure.

### TECHNICAL FIELD

This application relates generally to AI-based structure contouring using cross-attention segmentation.

### BACKGROUND

Radiotherapy (RT) is a critical treatment modality for cancer, involving the precise delivery of ionizing radiation to target malignant tissues while sparing healthy tissues as much as possible. The process begins with careful planning based on high-resolution imaging, typically using Computed Tomography (CT), and is followed by daily setup adjustments using Cone-Beam Computed Tomography (CBCT) to account for patient and tumor position changes. Despite advancements in imaging and treatment delivery, incorrect treatment positioning remains significant risks in radiotherapy. These errors can lead to the irradiation of non-target tissues or missing the target entirely, which can have severe consequences, including increased morbidity or failure to treat the cancer effectively.

Automated segmentation methods, including techniques that incorporate machine learning, seek to reduce delineation workload and unify the organ boundary definition. In deploying automated segmentation to clinical applications, however, segmentation and registration techniques generally require prior anatomic or geometric knowledge about the image content in order to work reliably. Machine learning systems, such as deep neural networks, are trained for auto-segmentation of anatomical organs with real images from clinical routine, such as computerized tomography (CT) images. These images normally do not cover entire patient body, rather, normally they cover a section of the “cranio-caudal range.” Therefore, training data normally includes image volume around a structure of interest, not full body images.

### SUMMARY

A problem that can arise when using machine learning for automated segmentation is that there may not be adequate training data available to train the machine learning model to segment every type of structure within the human body. For example, some structures within a human may be more commonly contoured than other structures. This may occur because contouring is typically performed to aid a radiotherapy machine in avoiding providing radiation to structures that are not relevant to treatment. Examples of such structures can include a pancreas and a spleen. As a result, there may not be an adequate amount of training data that is available to train a machine learning model to accurately contour the less commonly contoured structures.

A computing system can implement a few-shot learning technique using the spatial correspondence between structures in training images to train a machine learning model to contour less commonly represented structures. For example, the computing system can select a query medical image (e.g., a non-contoured medical image) and one or more support medical images (e.g., contoured medical images) of the same area or region of the human body. The computing system can generate an embedding representing or corresponding to spatial relationships between the same structures in each of the support medical images (e.g., as an average or median of locations of the structures within the support medical images) and/or the corresponding structures in the query medical image. The computing system can feed the query medical image, the support medical images, and the embedding into the machine learning model. The computing system can execute the machine learning model based on the input. Based on the execution, the machine learning model can use the spatial relationships represented by the embedding to propagate the contouring of the structures in the support medical images to the query medical image. The computing system can train the machine learning model based on the contoured query medical image. The computing system can repeat this process for any number of query medical images and corresponding support medical images to train the machine learning model over time. The computing system can subsequently use the trained machine learning model to contour new images using the spatial relationships between structures depicted in the new image to use to generate and/or use a radiotherapy treatment plan for treatment.

In an embodiment, a method may comprise generating, by at least one processor, a training dataset comprising a set of medical images comprising at least one query medical image depicting one or more structures within an anatomical region, and a plurality of support medical images depicting the one or more structures within the anatomical region, wherein the one or more structures are contoured and labeled; generating, by the at least one processor, a feature embedding for the at least one query medical image and the plurality of the support medical images, the feature embedding corresponding to a spatial correspondence between the one or more structures within the query medical image and the one or more structures within each support medical image; training, by the at least one processor, a machine learning model using the training dataset and the feature embedding, such that the machine learning model is configured to receive a new medical image and predict a contouring attribute for a first structure within the new medical image in accordance with a spatial correspondence to a second structure within the new medical image; and transmitting, by the at least one processor, the contouring attribute of the first structure to a radiotherapy computer model configured to generate a radiotherapy treatment plan for treatment of the first structure.

The machine learning model can be configured to predict the contouring attribute for the first structure within the new medical image based on a clinic-specific rule or a second rule specific to a medical professional.

The method can further include storing, by the at least one processor, a set of two-dimensional (2D) support medical images of one or more individuals in memory; and selecting, by the at least one processor, a subset from the set of 2D support medical that correspond to the anatomical region in the query medical image.

Selecting the subset of the set of 2D slices can include selecting, by the at least one processor, the subset of the set of 2D support medical images based on similarity scores between the query medical image and the subset of the set of 2D support medical images.

The method can further include generating, by the at least one processor, a confidence score for the contouring attribute based on the spatial correspondence between the first structure and the second structure in the new medical image.

Generating the confidence score can include generating, by the at least one processor, the confidence score based on an aggregation of pixel confidence scores for the contouring attribute of the new medical image.

The method can further include presenting, by the at least one processor, for display, the new medical image comprising a contour of the first structure.

One or more of the plurality of support medical images can depict a spatial relationship between the second structure and first structure, the plurality of support medical images each depicting a contour of the second structure.

The machine learning model can be configured to predict the contouring attribute for the first structure within the new medical image based on the spatial relationship between the second structure and the first structure in the one or more of the plurality of support medical images.

The method can further include retrieving, by the at least one processor, an inference set of medical images from a database based on similarities between the new medical image and the inference set of medical images, wherein the machine learning model is configured to generate the contouring attribute for the first structure based on the retrieved inference set of medical images.

One or more of the inference set of medical images can include a contouring of the first structure; and the machine learning model can be configured to generate the contouring attribute for the first structure in the new medical image based on a spatial correspondence between the first structure and the contouring of the first structure in the one or more of the inference set of medical images.

In another embodiment, a system may comprise a non-transitory computer-readable medium storing instructions that when executed cause at least one processor to generate a training dataset comprising a set of medical images comprising: at least one query medical image depicting one or more structures within an anatomical region, and a plurality of support medical images depicting the one or more structures within the anatomical region, wherein the one or more structures are contoured and labeled; generate a feature embedding for the at least one query medical image and the plurality of the support medical images, the feature embedding corresponding to a spatial correspondence between the one or more structures within the query medical image and the one or more structures within each support medical image; train a machine learning model using the training dataset and the feature embedding, such that the machine learning model is configured to receive a new medical image and predict a contouring attribute for a first structure within the new medical image in accordance with a spatial correspondence to a second structure within the new medical image; and transmit the contouring attribute of the first structure to a radiotherapy computer model configured to generate a radiotherapy treatment plan for treatment of the first structure.

The machine learning model can be configured to predict the contouring attribute for the first structure within the new medical image based on a clinic-specific rule or a second rule specific to a medical professional.

The instructions can further cause the processor to store a set of two-dimensional (2D) support medical images of one or more individuals in memory; and select a subset of the set of 2D images from the set of 2D support medical images that correspond to the anatomical region in the query medical image.

The instructions can cause the processor to select the subset of the set of 2D support medical images by selecting the subset of the set of 2D support medical images based on similarity scores between the query medical image and the subset of the set of 2D support medical images.

The instructions can further cause the processor to generate a confidence score for the contouring attribute based on the spatial correspondence between the first structure and the second structure in the new medical image.

The instructions can cause the processor to generate the confidence score by generating the confidence score based on an aggregation of pixel confidence scores for the contouring attribute of the new medical image.

The instructions can further cause the processor to present, for display, the new medical image comprising a contour of the first structure.

One or more of the plurality of support medical images can depict a spatial relationship between the second structure and first structure, the plurality of support medical images each depicting a contour of the second structure.

The machine learning model can be configured to predict the contouring attribute for the first structure within the new medical image based on the spatial relationship between the second structure and the first structure in the one or more of the plurality of support medical images.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates a diagram of a system for automatically contouring medical images, according to an embodiment.

FIG. 2 illustrates a flow diagram of a process for automatically contouring medical images, according to an embodiment.

FIG. 3 illustrates example support and query medical images, according to an embodiment.

FIG. 4 illustrates a sequence of images for automatically contouring of medical images, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are configured to be considered within the scope of the subject matter disclosed herein. Other embodiments can be used and/or other changes can be made without departing from the spirit or scope of the present disclosure. The illustrative embodiments described in the detailed description are not meant to be limiting of the subject matter presented.

Auto-contouring is an important part of the radiation therapy treatment planning process. Target volumes such as the gross tumor volume (GTV), clinical target volume (CTV), and planning target volume (PTV) can be contoured to delineate regions that will receive radiation treatment. Additionally, organs-at-risk (OARs) can be contoured to identify sensitive tissues that should be spared from high radiation doses. There are multiple contouring guidelines that provide protocols for target and OAR delineation, however, these guidelines can differ significantly across treatment sites.

While existing auto-contouring solutions can efficiently contour many commonly treated anatomical structures, there is still room for improvement before fully automated contouring can be achieved. One limitation is a lack of support for less frequently contoured OARs that may benefit from more precise dose sparing in certain scenarios. Another need is adapting to customized contouring styles, for example, the different acceptable ways physicians may contour the femoral head. Finally, variations in PTV delineation originating from the GTV, nearby lymph node CTVs, and CTV-to-PTV expansion margins are not fully automated.

Solving these problems, facilitating auto-contouring for uncommon OARs, incorporating customizable contouring protocols, and automating PTV delineation based on target volumes, would provide major steps toward enabling fully automated radiation therapy treatment planning. Further advances in auto-contouring will improve efficiency and consistency in the planning process across different treatment sites.

One type of auto-contouring is an atlas-based approach. This approach can involve collecting a number of curated atlas images with annotations. To contour a new image, registrations can be performed between each atlas and the new image and the annotations propagated from the atlas to the target space. Multi-atlas label fusion can then be used to obtain the final contour. In some cases, atlas selection can be performed between registration and label fusion so that the most similar atlases can be used for each specific new or target image. Such atlas-based approaches have the advantages of being easy to interpret (learning from templates), tending to preserve anatomical topology (although not guaranteed), and being adaptable to various contouring protocols (simply update atlases to contour in a new protocol). However, they also have disadvantages. For example, performance of some atlas-based approaches can be unsatisfactory, especially when atlases are dissimilar to the target image, and registration is time-consuming.

Emerging deep learning approaches have transformed auto-contouring by enabling improved performance and fast runtimes. One approach is to migrate from atlas-based to a convolutional neural network (CNN). This approach can result in the capability to automatically contour 153 anatomical structures, for example. Studies have shown auto-contouring via this approach can reduce contouring time by 75%. However, limitations remain with deep learning. It is data-driven, requiring huge efforts for data acquisition, curation, and annotation. This approach covers most common structures, but not less frequent ones or tumors (e.g., gross tumor volume (GTV), clinical target volume (CTV), planning target volume (PTV)). Additionally, many methods are deterministic, providing little contour adaptability to customized protocols. Users may find such auto-contouring techniques unacceptable if not contouring as desired, which can necessitate separate models for different protocols.

To overcome the aforementioned technical problems, a computer implementing the systems and methods described herein may facilitate training a machine learning model to use spatial relationships between structures depicted in an image to contour the structures in the image. The computer can train the machine learning model using a training data set that includes non-contoured query medical images and contoured support medical images matched to the query medical images. The computer can match support medical images to a query medical image based on the support medical images corresponding to the same region and/or having a high similarity with the query medical image. The machine learning model can be trained to propagate the contouring from the support medical images to the respectively matched query medical image based on an embedding representing a spatial correspondence and/or relationships between the structures depicted in the support medical images and/or the query medical image. The computer can similarly train the machine learning model using any number of matched query medical images and corresponding support medical images. In this way, the computer can train the machine learning model to contour structures of individuals based on the locations of the structures relative to the locations of other structures within the same images. The training can involve less training data than other training techniques because the training is more directed and more training (e.g., larger adjustments in weights) can be accomplished with each iteration of training (e.g., each iteration of training can involve spatial relationships between different structures that are relevant to future contouring of new images), which can increase the speed and efficiency of the training.

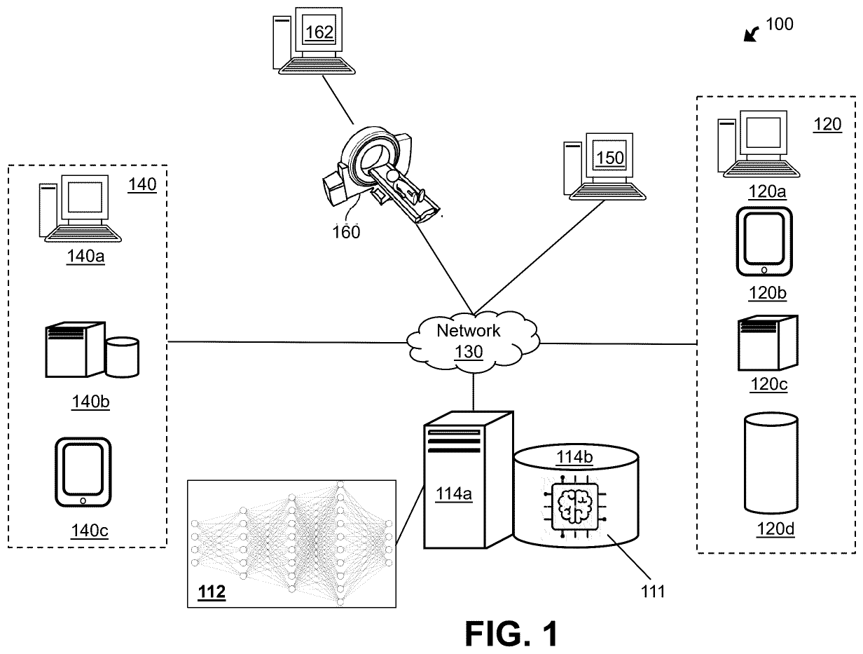

Subsequently, in an inference phase, the computer can use the trained machine learning model to generate or predict contouring attributes for structures based on the learned spatial relationships between the structures. In doing so, the machine learning model can predict contouring attributes for structures that are less commonly contoured with more accuracy. The trained model can allow for faster processing and contouring than other contouring methods because the processing may be based on fewer data points for contouring (e.g., instead of contouring structures based on each individual pixel of the structure, the machine learning model may only use the relationship between the objects depicted in the image for the contouring). FIG. 1 is a non-limiting example of components of a system in which such a computer (e.g., a server) operates.

FIG. 1 illustrates components of a system 100 for automatically contouring medical images, according to an embodiment. The system 100 can include an analytics server 114a, a system database 114b, a treatment planning system 111, electronic data sources 120a-d (each referred to individually as an electronic data source 120 and collectively electronic data sources 120, unless stated otherwise), end-user devices 140a-c (each referred to individually as an end-user device 140 and collectively as end-user devices 140, unless stated otherwise), an administrator computing device 150, a medical device 160, and medical device computer(s) 162.

Various components depicted in FIG. 1 can belong to a radiotherapy clinic at which patients can receive radiotherapy treatment, in some cases via one or more radiotherapy machines located within the clinic (e.g., medical device 160). The system 100 is not confined to the components described herein and can include additional or other components, not shown for brevity, which are configured to be considered within the scope of the embodiments described herein.

The above-mentioned components can be connected to each other through a network 130. Examples of the network 130 can include, but are not limited to, private or public local-area-networks (LAN), wireless LAN (WLAN) networks, metropolitan area networks (MAN), wide-area networks (WAN), and the Internet. The network 130 can include wired and/or wireless communications according to one or more standards and/or via one or more transport mediums. The communication over the network 130 can be performed in accordance with various communication protocols such as Transmission Control Protocol and Internet Protocol (TCP/IP), User Datagram Protocol (UDP), and IEEE communication protocols. In one example, the network 130 can include wireless communications according to Bluetooth specification sets or another standard or proprietary wireless communication protocol. In another example, the network 130 can also include communications over a cellular network, including, e.g., a GSM (Global System for Mobile Communications), CDMA (Code Division Multiple Access), and EDGE (Enhanced Data for Global Evolution) network.

The analytics server 114a can be any computing device comprising a processor and non-transitory machine-readable storage capable of executing the various tasks and processes described herein. The analytics server 114a can employ various processors such as central processing units (CPU) and graphics processing unit (GPU), among others. Non-limiting examples of such computing devices can include workstation computers, laptop computers, server computers, and the like. While the system 100 is shown to include a single analytics server 114a, the analytics server 114a can include any number of computing devices operating in a distributed computing environment, such as a cloud environment.

The analytics server 114a may execute software applications configured to display an electronic platform (e.g., host a website), which may generate and serve various webpages to each electronic data source 120 and/or end-user devices 140. Different users may use the website to view and/or interact with displayed content.

The analytics server 114a can generate and display the electronic platform, which may be configured to use the treatment planning system 111 for receiving patient information, inputs from users (e.g., clinicians) such as utility functions and updated utility functions described herein, and outputting the results of execution of the treatment planning system 111. The electronic platform can include graphical user interfaces (GUI) displayed by display devices of one or more electronic data sources 120, the end-user devices 140, the medical device 160, and/or the administrator computing device 150. An example of the electronic platform generated and hosted by the analytics server 114a can be a web-based application or a website configured to be displayed on different electronic devices, such as mobile devices, tablets, personal computers, and the like.

The analytics server 114a may use one or more machine learning models 112 for AI-based atlas mapping slice localization to obtain training data, and for training deep learning models using the training data. More specifically, at an inference phase for a patient radiotherapy treatment plan, the electronic platform may display one or more medical images, such as images of patient OARs or other internal organs, and images of auto-segmentation contours of such organs.

Additionally, the electronic platform may display two-dimensional and/or three-dimensional plots output by the machine learning models 112 based upon atlas mapping slice localization, and based upon auto-segmentation contours of the prior patient's medical images and the current patient's medical images.

In a non-limiting example, a physician or radiation oncologist operating the medical professional device 120b may access the platform, review the results of atlas mapping slice localization, and review auto-segmentation contours. The physician or radiation oncologist may visually inspect two-dimensional and/or three-dimensional plots based upon atlas mapping slice localization and based upon auto-segmentation contours of prior patient and current patient medical images. The medical professional devices (e.g., the medical professional device 140c) may be used as both a device to display results predicted by the analytics server 114a and in some cases as an electronic data source (e.g., electronic data source 120b) to train the machine learning model 112.

The electronic data sources 120 can each be any computing device comprising a processor and non-transitory machine-readable storage capable of executing the various tasks and processes described herein. For example, the electronic data sources 120 can represent various computing devices that contain, retrieve, and/or access data associated with a medical device 160, such as data associated with operational information of currently or previously performed radiotherapy treatments (e.g., electronic log files or electronic configuration files), data associated with current and/or previously monitored patients (e.g., computed tomography (CT) scans, magnetic resonance imaging (MRI) scans, tumor locations, deformation information, and/or the like) or participants in a study, and/or the like. For instance, the analytics server 114a can use the clinic computer 120a, medical professional device 120b, server 120c (associated with a clinician and/or a clinic), and database 120d (associated with the clinician and/or the clinic) to retrieve/receive data associated with the medical device 160. The analytics server 114a can retrieve the data from the end-user devices 140, generate a dataset, and use the dataset to configure the treatment planning system 111 (e.g., models implemented by the treatment planning system 111 and/or the like). The analytics server 114a can execute various algorithms to translate raw data received/retrieved from the electronic data sources 120 into machine-readable objects that can be stored and processed by other analytical processes as described herein.

The analytics server 114a may receive medical images from a user or retrieve such data from a data repository, analyze the data, and display the results on the electronic platform. For instance, in a non-limiting example, the analytics server 114a may query and retrieve medical images from the database 120d and combine the medical images with segment or contouring data received from a physician operating the medical professional device 120b. Additionally, or alternatively, the analytics server 114a may segment or contour the medical image automatically or perform other pre-processing steps on the medical image captured from the medical device 160.

End-user devices 140 can be any computing device comprising a processor and a non-transitory machine-readable storage medium capable of performing the various tasks and processes described herein. Non-limiting examples of an end-user device 140 can be a workstation computer, laptop computer, tablet computer, or server computer. In operation, various users such as clinicians as described herein can use end-user devices 140 to access the GUI operationally managed by the analytics server 114a or otherwise the results of the execution of the treatment planning system 111. Specifically, the end-user devices 140 can include a clinic computer 140a, a clinic server 140b, and a medical professional device 140c.

Even though referred to herein as “end-user” devices, these devices may not always be operated by end-users. For instance, the clinic server 140b may not be directly used by an end user. However, the results stored on the clinic server 140b may be used to populate various GUIs accessed by an end user via the medical professional device 140c. In some embodiments, the end-user device 140 can be associated with one or more clinicians that are associated with the generation of one or more treatment plans (e.g., involved in preparing the one or more treatment plans) for patients.

The administrator computing device 150 can represent a computing device operated by a system administrator. The administrator computing device 150 can be configured to display radiotherapy treatment attributes generated by the analytics server 114a (e.g., various analytic metrics determined during training of one or more machine learning models and/or systems); monitor various treatment planning systems 111 utilized by the analytics server 114a, electronic data sources 120, and/or end-user devices 140; review feedback; and/or facilitate training or retraining (calibration) of the treatment planning system 111 that are maintained by the analytics server 114a. In some embodiments, the methods and systems discussed herein may utilize transfer learning paradigms, such as by adapting a pre-trained machine learning model, originally trained on a broad dataset, using/leveraging the learned knowledge of the models discussed herein.

In some embodiments, the medical device 160 can be a diagnostic imaging device, a radiotherapy machine, or a treatment delivery device. For example, the medical device 160 can include one or more computed tomography (CT) scanners, linear accelerators (LINACs) having a multi-leaf collimator (MLC) that consists of multiple small lead leaves that can be individually moved to shape the radiation beam and deliver the dose to the tumor while minimizing the dose to surrounding healthy tissues, or other similar devices configured to transmit energy toward targeted tissue (referred to as planning target volumes) associated with a patient and, in some cases, measure the energy transferred to ward the targeted tissue. In some embodiments, the medical device 160 can receive data associated with a treatment plan from the medical device computer(s) 162 that cause the medical device 160 to operate in accordance with the treatment plan.

The treatment planning system 111 can be configured to generate radiotherapy treatment plans to treat structures (e.g., anatomical structures, such as organs, bones, tumors, etc.) of individuals. The treatment planning system 111 can be stored in the system database 114b and/or stored by a computing device remote from the analytics server 114a. The treatment planning system 111 can be or include a radiotherapy computer model trained using data received/retrieved from the electronic data sources 120 and can be executed using data received from the end-user devices 140 and/or the medical device 160. In some embodiments, the treatment planning system 111 can reside within a data repository local or specific to a clinic. In various embodiments, the treatment planning system 111 can use one or more deep learning engines to develop a treatment plan for a patient using radiation therapy. For instance, the analytics server 114a can transmit patient attributes and/or contouring attributes (e.g., labels indicating edges and/or types of structures depicted in an image) to the treatment planning system 111 and execute the treatment planning system 111 accordingly. The analytics server 114a can then display the results on one or more end-user devices 140. In some embodiments, the analytics server 114a can change one or more configurations of the medical device 160 based on the results predicted by the treatment planning system 111.

In operation, a physician or other medical professional may access an application executing on the medical professional device 120b and input patient data and the patient's treatment data (e.g., patient information, patient diagnosis, radiation therapy radiation requirements and thresholds). The analytics server 114a then uses a patient identifier to query patient data (e.g., patient anatomy and/or medical images) from the electronic data sources 120. The analytics server 114a may then identify a clinic associated with the patient (e.g., the clinic performing the treatment) and retrieve one or more files associated with treatment templates and clinic rules. The analytics server 114a may utilize the systems and methods described herein to generate AI-based atlas mapping slice localization data based on input patient images.

A medical professional at a radiotherapy clinic may access an end-user device 140 located at the clinic or access an account associated with the clinic. The medical professional may provide an input at a user interface that causes the end-user device 140 to transmit a request to access a machine learning model 112 that is associated with the clinic and/or the radiotherapy machines located within the clinic. The request may include an identifier associated with the machine learning model 112, the clinic, a treatment plan generated by the one or more medical professionals, and/or the set of radiotherapy machines that the analytics server 114a may use as a key in a look-up table to identify the machine learning model 112. The analytics server 114a may receive the request and, in some cases, after authenticating the user, identify the machine learning model 112 via the identifier. The analytics server 114a may transmit the identified machine learning model 112 to the end-user device 140 or send an alert indicating the end-user device 140 is authorized to access the model(s) 112. Upon receipt or access to the machine learning model 112, the end-user device 140 may perform the systems and methods described herein to train the machine learning model 112.

The analytics server 114a may store machine learning models 112 (e.g., neural networks, random forest, support vector machines, or other deep learning models). The machine learning model 112 may be trained to predict the anatomical structure represented by various pixels or voxels of a medical image. Various machine learning techniques may involve training the machine learning models to predict (e.g., estimate the likelihood of) each pixel or voxel of a medical image being associated with or otherwise representing a particular anatomical structure, or excluding a particular anatomical structure, based on outputs of AI-based atlas mapping slice localization.

Machine learning models 112 may be stored in the system database 114b and may correspond to individual radiotherapy clinics or otherwise different sets of radiotherapy machines (e.g., radiotherapy machines that are located at individual radiotherapy clinics, are located in different geographical regions, treat specific types of diseases (e.g., different types of cancer), treat specific genders, etc.). For example, the machine learning model 112 may be associated with an identifier indicating the radiotherapy clinic, set of radiotherapy machines, or a specific disease.

In various embodiments, the machine learning models 112 use one or more deep learning engines to perform automatic segmentation of image data for radiotherapy treatment planning. Although exemplified using deep convolutional neural networks, it should be understood that any alternative and/or additional deep learning model(s) may be used to implement deep learning engines. The deep learning engines include processing pathways that are trained during a training phase. Once trained, deep learning engines may be used (e.g., by a clinician) to perform automatic segmentation for current patients during inference phase.

In the training phase, the machine learning model 112 may be trained as a deep learning engine to perform automatic classification of input segmentation data by mapping the input data (segmentation data) to example output data (labels). The training phase may involve identifying or determining weights that minimize the training error between training label data and estimated label data generated by a deep learning engine. The analytics server 114a may apply the two-dimensional (2D) anatomical landmarks method to train a deep learning model using data labeled via this method (e.g., ground truth detection). Once the training data is labeled, the deep learning model learns visual attributes of a certain anatomical region and can identify numerical values for the anatomical region that correspond to localized slices within an atlas or reference image (e.g., CT scan). In practice, by training a contouring model using data labeled via 2D anatomical landmarks method, if a contouring machine learning model knows that an input CT is taken from a particular patient anatomic region, the contouring machine learning model does not need to run an analysis that is specific to another anatomic region. In another example, deep-learning contouring models may be trained for specific anatomical regions. For example, one model may be specially trained to identify organs in a patient's head and neck.

The analytics server 114a can train the machine learning model 112 for segmentation (e.g., two-dimensional and/or three-dimensional segmentation) using a few-shot learning approach. The analytics server 114a can train the machine learning model 112 using a cross-attention technique in which the analytics server 114a can match individual query medical images depicting one or more structures within an anatomical region of an individual (e.g., an image depicting an overhead “slice” of an anatomical region of an individual) to one or more support medical images that depict the same one or more structures within the anatomical region. The one or more structures can be contoured and/or labeled in the one or more support medical images (e.g., in each of the one or more support medical images). The query medical image may not be contoured.

The analytics server 114a can feed the query medical images and corresponding matched support medical images into the machine learning model 112 and execute the machine learning model 112. In some cases, for each query medical image, the analytics server 114a can additionally generate and include a feature vector identifying or corresponding to spatial relationships between the structures in the respective query medical images and/or support medical images (e.g., as an average or median of embeddings generated for each of the support medical images) matched to the query medical images The spatial relationships can be distances (e.g., number of pixels or voxels) and/or directions of distances between structures depicted in the respective images. For each of the query medical images, the analytics server 114a can execute and/or cause the machine learning model 112 to contour or label the pixels or voxels of the query medical image that represent different structures depicted in the query medical image based on the labels for the same structures that are depicted in the support medical images and the feature vector indicating the spatial correspondence between the structures depicted in the respective images.

For example, the machine learning model 112 can use matching techniques (e.g., semantic matching techniques) to identify common regions within the query and support medical images. The machine learning model 112 can identify any labels or contours of the support medical images in the identified common regions and use the feature vector identifying the spatial correspondence between the structures depicted in the query medical image and the matched support medical images to propagate or otherwise label the common regions in the query medical image with the same labels. The analytics server 114a can then use a loss function and/or backpropagation techniques to train (e.g., adjust the weights of) the machine learning model 112 against a ground truth labeled version of the query medical image. The analytics server 114a can repeat this process for any number of query and corresponding support medical images. By doing so, the analytics server 114a can train the machine learning model 112 to use the spatial correspondence between structures depicted in individual images to more accurately contour images, particularly for structures of individuals for which there is less available training data.

In a non-limiting example, the analytics server 114a can train the machine learning model 112 to contour structures of individuals depicted in images based on a spatial correspondence between the respective structures (e.g., a number of pixels or voxels and/or the direction of the distances between the structures) as depicted in the images. To do so, the analytics server 114a can generate a training dataset comprising a set of medical images. The set of medical images can include at least one query image or query medical image (e.g., a non-contoured or non-labeled image that the machine learning model 112 will contour in performing this process) and one or more (e.g., a plurality of) support images or support medical images (e.g., contoured or labeled images that the machine learning model 112 can use to label the query medical image). The query medical image can depict one or more structures within an anatomical region of an individual (e.g., in a top view). The support medical images can depict the one or more structures within the anatomical region (e.g., the same one or more structures and anatomical region to the query medical image). For instance, the query medical image and the support medical images can depict different slices (e.g., a two-dimensional or three-dimensional slice) of the same anatomical region of the individual in a top view. The query medical image and the support medical images may be matched together to use for training the machine learning model 112 based on the respective images depicting the same anatomical region and/or structures. The analytics server 114a can generate the training dataset to include any number of such matched query medical images and matched support medical images.

The analytics server 114a can generate a feature embedding indicating the spatial correspondences between respectively matched query and support medical images within the training dataset. The spatial correspondences can be the relationships between different structures depicted in each of the support medical images and/or the query medical image. The analytics server 114a can generate the feature embedding using feature extraction techniques, for example. For instance, for a matched query medical image and corresponding support medical images, the analytics server 114a can extract features (e.g., colors, edges, boundaries, text descriptors, semantic features, contrast, scene, etc.) from the query medical image and support medical images. The analytics server 114a can do so using one or more convolutional neural networks (CNNs) or any other technique. After extracting the features, the analytics server can identify matching features between the different images, or features that otherwise semantically correspond with each other between the different images. The analytics server can identify the matching features using feature matching techniques, such as by comparing the features and identifying matching values or by using nearest-neighbor searches. The analytics server 114a can use nearest-neighbor searches enhanced by algorithms such as random sample consensus (RANSAC). In some cases, the analytics server 114a can generate the embedding by generating separate embeddings for each of the support medical images and calculating an average or median of the embeddings of the support medical images, thus creating an embedding of the average or median locations of the structures depicted in the support medical images matched to the query medical image.

In some cases, to align the spatial information across images, the analytics server 114a can implement geometric transformations or attention mechanisms. In doing so, the analytics server 114a can ensure that the relative positions of objects depicted are consistently represented for the matching. The analytics server 114a can combine the aligned or matched features between images, such as through concatenation or pooling. The analytics server 114a can pass the combined or matched features through dimensionality reduction layers to form a unified embedding. Throughout this process, the analytics server 114a can use loss functions that preserve spatial relationships. Thus, corresponding objects across different images can be closely situated within the embedding space. The generated embedding can encode the spatial correspondences between structures depicted within each of the images and/or between the respective images. The analytics server 114a can similarly generate such embeddings for any number of query medical images matched to support medical images.

The analytics server 114a can train the machine learning model 112 using the training dataset and the generated embeddings. For example, for each query medical image, the analytics server 114a can feed the query medical image into the machine learning model 112 with one or more support medical images matched to the query medical image and an embedding corresponding to (e.g., representing) the spatial correspondence between the structures of the query medical image and the structures of the support medical images. The analytics server 114a can execute the machine learning model 112 to cause the machine learning model 112 to use the embedding to map or otherwise identify locations within the query medical image that correspond to locations of the support medical images that depict structures within the individual. The depicted structures (e.g., the pixels or voxels depicting the structure) within the support medical images may be labeled to indicate the structures and/or the type of structure (e.g., organ or type of organ, tumor, bone, etc.). Based on the identification of the locations, the machine learning model 112 can propagate the labels for the structures from the support medical images to the corresponding locations (e.g., pixels or voxels) in the query medical image. The analytics server 114a can additionally or instead update the weights of the machine learning model 112 to make it more likely that the machine learning model 112 would generate a similar contouring attribute or contour of the query medical image if the machine learning model 112 were to process the query medical image alone and/or again. The analytics server 114a can similarly train the machine learning model 112 using any number of query medical images matched to support medical images and corresponding embeddings. In doing so, the analytics server 114a can train the machine learning model 112 in a training phase using a few-shot learning technique to label or contour structures depicted in medical images.

In some cases, during the training phase, the machine learning model 112 can learn patterns of locations of structures relative to other structures within the same images. For example, the feature embedding indicating the spatial correspondence between structures in the images can indicate the locations of the structures depicted in the images relative to each other. By using the feature embedding to contour a query medical image and then training the machine learning model 112 based on the contoured query medical image, the machine learning model 112 may learn parameters and/or patterns of locations of structures relative to each other. Thus, when the machine learning model 112 is subsequently used for inference, the machine learning model 112 may be able to accurately identify and contour less commonly contoured structured using the learned patterns and/or parameters from spatial relationships the structures with other structures depicted in the same images.

In an inference phase, the machine learning model 112 can process new images to predict contouring attributes (e.g., contour) for structures within the new images. The analytics server 114a can contour the images using the machine learning model 112 responsive to determining the machine learning model 112 is sufficiently trained (e.g., is accurate above an accuracy threshold). For example, the analytics server 114a can obtain a new medical image. The analytics server 114a can obtain the new medical image by receiving the medical image from a remote computing device and/or by retrieving the medical image from a database (e.g., the database 120d). The new medical image may depict a plurality of structures that were depicted in a query medical image and corresponding support medical images. The analytics server 114a can feed or input the new medical image into the machine learning model 112 and execute the machine learning model 112 based on the input. Based on the execution, the machine learning model 112 can apply the weights learned during the training phase on the spatial correspondence between the structures depicted in the new image to contour the respective structures within the new image. In some cases, in doing so, the machine learning model 112 may contour the new image by applying weights learned contouring images of the same anatomical region and/or set of structures of one or more training images as depicted in the new image. In this way, the analytics server 114a can implement a few-shot learning technique using a spatial correspondence between structures depicted in an image to contour the structures in the image, which can facilitate training the machine learning model 112 using less training data and/or more targeted training data.

The analytics server 114a can transmit the contouring attributes predicted or generated by the machine learning model 112 to the treatment planning system 111 (e.g., the radiotherapy computer model). The analytics server 114a can transmit the contouring attributes to the treatment planning system 111 by contouring the new image and transmitting the contoured new image to the treatment planning system 111 and/or by transmitting values or indications of labels or indications of the structures within the patient to the treatment planning system 111. The analytics server 114a can transmit the contouring attributes to the treatment planning system 111 by transmitting the contouring attributes across the network 130 to another computer or computer system hosting the treatment planning system 111 or by transmitting the contouring attributes to a location in memory of the analytics server 114a hosting the treatment planning system 111.

The treatment planning system 111 can use the transmitted contouring attributes to generate a radiotherapy treatment plan for treatment of a structure depicted within the new image. For example, the treatment planning system 111 can use the contouring attribute to generate a treatment plan that includes treatment attributes, such as different treatment modalities, field geometry settings for external beam radiotherapy, side effect predictions, organ and/or tumor segmentation, machine therapy attributes, dosage administration attributes (e.g., dosage amount), treatment frequency, treatment timing, monitored units, etc. For instance, the treatment planning system 111 can use the contouring attribute to generate field geometry settings in the radiotherapy treatment plan including beam angles, multi-leaf collimator positions, and/or gantry rotations to either target a particular contoured structure with radiation or avoid the contoured structure, such as if the contoured structure is an organ-at-risk. The treatment planning system 111 may additionally or instead use the contouring to determine a dosage amount to use to use when treating a particular structure. The treatment planning system 111 can generate the radiotherapy treatment plan to include such attributes such that a radiotherapy machine learning can treat patient and/or avoid providing too high of a dosage of radiation to an organ-at-risk.

The analytics server 114a can use the radiotherapy treatment plan to treat the individual. For instance, the analytics server 114a can configure or control the medical device 160 according to the radiotherapy treatment plan generated using the contouring attributes of the new image. The analytics server 114a can activate or control the medical device 160 according to the treatment attributes of the radiotherapy treatment plan to treat the patient. Thus, the analytics server 114a can use the machine learning model 112 to generate a radiotherapy treatment plan to treat the individual.

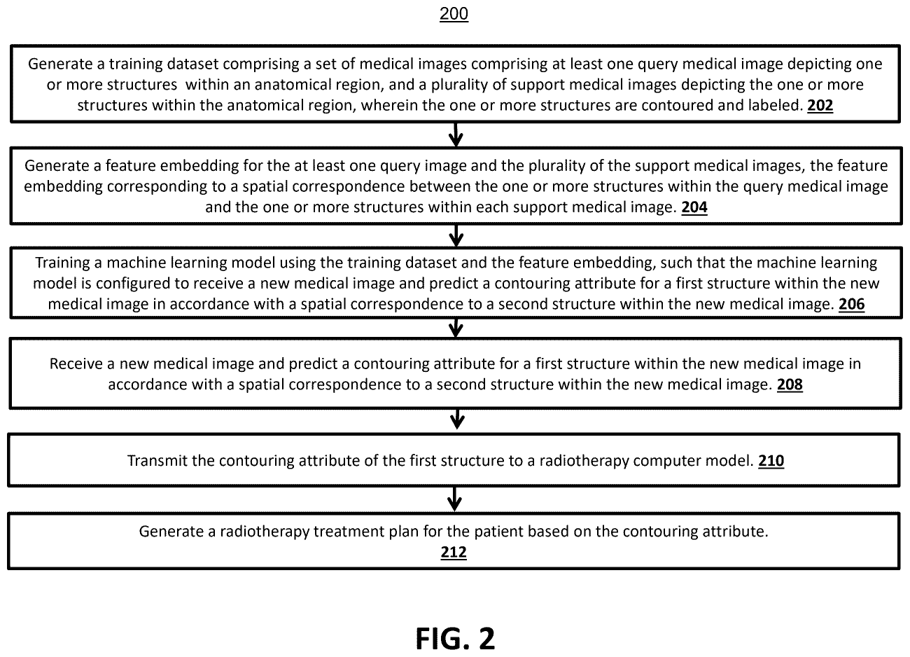

Referring to FIG. 2, illustrated is a flow diagram of a method 200 for automatically contouring medical images, according to an embodiment. The method 200 includes operations 202-212. However, other embodiments can include additional or alternative operations or can omit one or more operations altogether. The method 200 is described as being executed by an analytics server, which can be the same as, or similar to, the analytics server 114a described in FIG. 1. However, one or more steps of the method 200 can be executed by any number of computing devices operating in the distributed computing system described in FIG. 1. For instance, one or more computing devices can locally perform part or all of the operations described in FIG. 2.

By implementing the method 200, the analytics server may employ a few-shot training technique in which the analytics server trains a machine learning model to contour new images based on spatial relationships between structures depicted in the images. The analytics server can do so by matching individual query medical images to one or more support medical images of structures of an individual. The support medical images can be labeled or contoured. The analytics server can generate an embedding representing the spatial correspondence between the structures depicted in the query medical images and the respectively matched support medical images. The analytics server can feed the individual query medical images with the matched labeled or contoured support medical images with the corresponding embeddings into a machine learning model to train the machine learning model to contour images based on spatial relationships between structures depicted in the images. The analytics server can then use the trained machine learning model to contour (e.g., generate a contouring attribute for) a new image based on the spatial relationships between the structures depicted in the new image. The analytics server can use the contouring of the new image to generate a radiotherapy treatment plan for patients through a radiotherapy computer model. The analytics server can configure and/or a radiotherapy machine according to the radiotherapy treatment plans to treat the patients. As used herein, a spatial relationship is another term for a spatial correspondence, and vice-versa.

At step, 202, the analytics server may generate a training dataset. The analytics server can generate the training dataset to include a set of medical images. The set of medical images can at least include a query medical image depicting one or more structures within an anatomical region and a plurality of support medical images depicting the one or more structures within the anatomical region. The structures can be labeled within the images, in some cases, only in the support medical images and not the query medical image. The structures can be or include different anatomical structures within a body, such as organs or bones. The medical images can be or include one or more of computed tomography (CT), cone beam computed tomography (CBCT), X-ray, magnetic resonance imaging (MRI), ultrasound, positron emission tomography scan (PET) scan, single photon emission computed tomography (SPECT), tomosyntheses, and/or fluoroscopy images of cross-sections of one or more patients.

The images of the training dataset can be images of “slices” of an individual body. For example, the images can be two-dimensional or three-dimensional images at a cross-section of an individual body in a top view. For instance, the images can depict an area or region of an individual's body when looked at from above.

The analytics server can match support medical images to query medical images to include in the training dataset. The analytics server can do so, for example, based on the support medical images and/or query medical images corresponding to the same anatomical region and/or having a similarity with each other above a threshold. For instance, for individual patients, the analytics server can store sets of images (e.g., two-dimensional or three-dimensional images) that depict different cross-sections of the patient's body. One or more, or all, of the stored sets of images can be annotated or labeled with contouring of the images to outline the structures depicted in the images, in some cases with indications of the types of the respective structures. The images can depict the spatial relationships between the structures (e.g., the amount of space or number of pixels or voxels between the different structures) depicted in the images. The annotations may have been performed manually or automatically prior to being stored by the analytics server. In some cases, the individual images can be stored with labels indicating the anatomical regions (e.g., chest, waist, stomach, head, leg, etc.) that are depicted in the respective images.

The analytics server can identify a query medical image (e.g., a non-annotated image) of a particular anatomical region of a patient, such as from the stored sets of images or by receiving the query medical image from a remote computing device. The analytics server can determine an anatomical region of the query medical image, such as based on an indication of the anatomical region in a message containing the query medical image from the remote computing device, based on a label of the query medical image stored in memory, or by using object recognition techniques (e.g., by using a machine learning model trained to determine anatomical regions for images based on the objects depicted in the images) on the query medical image to determine the anatomical region of the query medical image. The analytics server can retrieve a subset of support medical images (e.g., annotated medical images) of the set of images of patients stored in memory responsive to determining each image of the subset matches or otherwise corresponds to the same anatomical region as the query medical image (e.g., by comparing the determined anatomical region of the query medical image with the labels of the support medical images indicating the anatomical regions the support medical images depict). The analytics server can include the query medical image and the subset of support medical images in the training dataset to use to train a machine learning model to contour structures.

In some cases, in addition to or instead of using labels of anatomical regions to identify support medical images that match to a query medical image, the analytics server can generate or determine similarity scores between the query medical image and the individual stored images. For example, the analytics server can use machine learning techniques to generate a similarity score between the query medical image and each of the individual stored images. To do so, the analytics server can execute an embedding machine learning model (e.g., an encoder or neural network) configured or trained to generate embeddings in an embedding space of images to generate a query medical image embedding (e.g., a vector or numerical vector with numbers at different index values representing features depicted in the query medical image) of the query medical image. The analytics server can similarly execute the embedding machine learning model to generate a support medical image embedding for each of the support medical images stored in memory. The analytics server can compare the query medical image embedding with the support medical image embeddings using matching or cosine similarity techniques to generate similarity scores between the query medical image embedding and the support medical image embeddings. The analytics server can compare the similarity scores with each other and identify the support medical images that correspond with a defined number (e.g., one, two, three, or any number) of the highest similarity scores and/or that satisfies (e.g., exceeds) a threshold to identify the support medical images that match to the query medical image. The analytics server can include the identified matching support medical images in the training data set.

In some cases, the analytics server can use a combination of the labels and similarity scores to select support medical images that match to a query medical image. For example, when creating the training dataset, the analytics server may first retrieve potential support medical images to include in the training dataset with the query medical image based on the potential support medical images corresponding to the same anatomical region as the query medical image. Subsequent to retrieving the potential support medical images, the analytics server can generate similarity scores for the retrieved potential support medical images (e.g., only the retrieved potential support medical images) and select support medical images that match the query medical images based on the similarity scores as described above. In doing so, the analytics server can both ensure that the support medical images included in the training dataset are relevant for contouring the query medical image and do so without using the processing power required to compare the embedding for the query medical image with the embedding for each stored support medical image, which can substantially reduce latency and the computational resources involved in creating the training dataset and training the machine learning model for contouring.

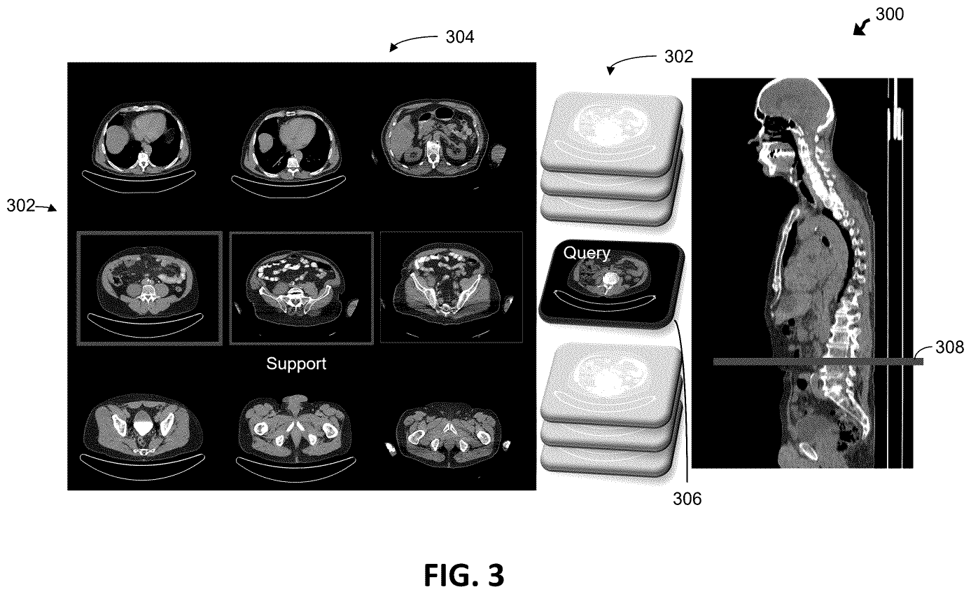

For example, referring now to FIG. 3, example query and support medical images 300 are depicted, according to some embodiments. The query and support medical images 300 can be stored in memory or a database of the analytics server and/or the analytics server can receive the query and support medical images 300 from a remote computing device. The example query and support medical images 300 can include query medical images 302 and support medical images 304. The analytics server can retrieve or receive a query medical image 306 to include in the training dataset. The analytics server can determine the query medical image depicts a mid-section anatomical region based on a label stored or included in a message containing the query medical image 306 and/or by using object recognition techniques on the query medical image 306. The analytics server can select a subset 310 of the set of support medical images 304 based on the subset 310 of support medical images depicting the same anatomical region 308 as the query medical image 306 and or based on the subset 310 of support medical images having a similarity score exceeding a threshold and/or having a highest similarity score with the query medical image 306 of the set of support medical images 304.

Selecting appropriate support medical images for individual query medical images can be important, as it directly impacts segmentation performance during the training phase. Given numerous potential support medical images, the analytics server may retrieve the support medical images that are the most similar to the respective query medical images. This differs from conventional active learning techniques, which can focus on representativeness and diversity. In the context of few-shot learning, high similarity between the query and support medical images can facilitate simplified segmentation approaching direct mask propagation. Conversely, support medical images depicting irrelevant objects may not be informative.

In some embodiments, the analytics server can establish correspondences between query and potential support medical images at both image and region levels. For example, the analytics server can use contrastive techniques, such as a Medical Localize and Segment Anything Model (MedLSAM), to define positive and negative pairs during training. The analytics server can then use a similarity-based image retrieval process with an embedding of the query medical image to identify the support medical images that are the most similar to the query medical image. If the retrieved support medical images are already annotated, the labels can be used by the machine learning model to directly propagate labels from the support medical images to the query medical image. Otherwise, the non-annotated support medical images may be annotated to expand the few-shot support medical image set. Performing these processes using locality-sensitive hashing of embeddings and approximate nearest-neighbor search can facilitate efficient retrieval.

Referring again to FIG. 2, the query medical image and the support medical images may each correspond to the same individual or may correspond to different individuals. For example, the query medical images may be an image of an anatomical region of one individual and each of the support medical images may be an image of the anatomical region of a different individual from the query medical images and/or from each other. Using images from different individuals may increase the training data that is available for the training dataset and/or provide a more diverse training dataset that can improve the accuracy of the machine learning model for training for different patients, for example. Using training data from the same individual can improve the accuracy of contouring structures of a specific individual, which can be useful when the analytics server is using the machine learning model to contour images of structures of an individual based on which the machine learning model used medical images for training.

The analytics server can similarly match individual query medical images to one or more support medical images for any number of query medical images. The analytics server can include matched query medical images and support medical images in a training dataset to generate or create the training dataset.

At step 204, the analytics server may generate a feature embedding for each query medical image and corresponding matching plurality of support medical images (e.g., each set of query medical image and corresponding matching support medical images). The feature embedding can correspond or otherwise represent a spatial correspondence between the structures depicted in the query medical image and/or the structures depicted in the support medical images. The analytics server may generate the feature embedding for a query medical image and matched support medical images, for example, using feature extraction techniques. For instance, the analytics server can extract features (e.g., colors, edges, boundaries, text descriptors, semantic features, contrast, scene, etc.) from the query medical image and support medical images. The analytics server can do so using one or more convolutional neural networks (CNNs) or any other technique. In some cases, the analytics server can generate the feature embedding only from the support medical images (e.g., by only extracting features from the support medical images matched to the query medical image).

After extracting the features from the query medical image and/or the support medical images (e.g., only the support medical images), the analytics server can identify matching features between the different images, or features that otherwise semantically correspond with each other between the different images. The analytics server can identify the matching features using feature matching techniques, such as by comparing the features and identifying matching values or by using nearest-neighbor searches. The analytics server can use nearest-neighbor searches enhanced by algorithms such as random sample consensus (RANSAC) to ensure robustness against outliers. In some cases, to align the spatial information across images, the analytics server can implement geometric transformations or attention mechanisms. In doing so, the analytics server can ensure that the relative positions of objects depicted are consistently represented for the matching. The analytics server can combine the aligned or matched features between images, such as through concatenation or pooling. The analytics server can pass the combined or matched features through dimensionality reduction layers to form a compact, unified single embedding. Throughout this process, the analytics server can use loss functions that preserve spatial relationships. Thus, corresponding objects across different images can be closely situated within the embedding space. The generated embedding can encode (e.g., include numerical values or representations of) the spatial correspondences between structures depicted within the images. The analytics server can similarly generate such embeddings for any number of query medical images matched to support medical images. The analytics server can similarly generate such feature embeddings for each set of query medical image and/or matching support medical images.

In some cases, the analytics server can generate (e.g., using an encoder or embedding machine learning model) separate feature embeddings for each support medical image matched to the query medical image. The analytics server can generate the embeddings in the same embedding space by using the same model to generate each embedding. The separate feature embeddings can indicate or represent the spatial correspondence between the structures depicted in each of the images.

In some cases, the analytics server can generate an aggregate embedding for (e.g., only for) the support medical images matched to the query medical image. For example, the analytics server can generate individual embeddings (e.g., using an embedding machine learning model) for each of the support medical images. The analytics server can generate or calculate an average or median embedding from the generated embeddings. In doing so, because the support medical images each correspond to the same region and the region of the query medical image, the average embedding can indicate an average spatial correspondence between structures of the region depicted within the query medical image.

At step 206, the analytics server may train the machine learning model (e.g., a neural network, a support vector machine, a random forest, a transformer, a generative machine learning model, etc.) using the training dataset and the feature embedding for each query medical image and corresponding matching support medical images. For example, for each query medical image of the training dataset, the analytics server can input the query medical image, matching support medical images, and feature embedding corresponding to (e.g., representing) the spatial correspondence between the one or more structures depicted in the query medical image and the one or more structures depicted in the support medical images into the machine learning model. The analytics server can execute the machine learning model to cause the machine learning model to use the feature embedding to map or otherwise identify locations within the query medical image that correspond to locations of the support medical images that depict structures. The depicted structures (e.g., the pixels or voxels depicting the structure) within the support medical images may be labeled to indicate the structures and/or the type of structure (e.g., organ or type of organ, tumor, bone, etc.). Based on the identifications of the locations, the machine learning model can use the feature embedding to propagate the labels for the structures from the support medical images to the corresponding locations (e.g., pixels or voxels) in the query medical image. The analytics server can additionally or instead update the weights of the machine learning model to make it more likely that the machine learning model would generate a similar contouring attribute or contour of the query medical image if the machine learning model were to process the query medical image alone and/or again. The analytics server can similarly train the machine learning model using any number of query medical images matched to support medical images and corresponding embeddings. In doing so, the analytics server can train the machine learning model in a training phase using a few-shot learning technique to label or contour structures depicted in medical images.

The analytics server may perform the steps 202-206 of the method 200 by segmenting a query medical image by leveraging a defined number (e.g., a number, N) of annotated support medical images. The analytics server can use a pre-trained matching network that uses cross-attention between a query medical image and the support medical images for few-shot learning. The analytics server can use a cross-attention module to build an understanding of semantic correspondences between the query and support medical images. Doing so can facilitate robustness against variations in anatomical structures, image contrasts, artifacts, and different modalities when trained. Specifically, the cross-attention can identify related regions between the query and most relevant support medical images. These correspondences can allow propagating the support medical images segmentations to appropriately match the query medical image.

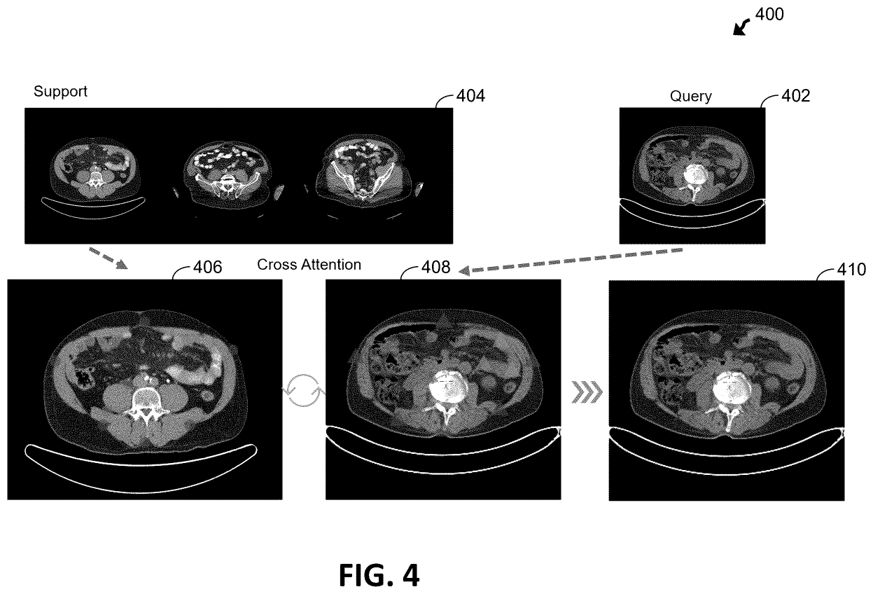

For example, referring now to FIG. 4, a sequence 400 of using a support medical images to contour a query medical image is shown, according to some embodiments. In the sequence 400 a training dataset can include a query medical image 402 and support medical images 404 matched to the query medical image 402. The analytics server can input the query medical image 402, the support medical images 404, and a feature embedding corresponding to the spatial correspondence between the one or more structures depicted in the query medical image and the one or more structures depicted in the support medical images into the machine learning model. The analytics server can execute the machine learning model to identify regions of the support medical regions (depicted with circle and triangle markers in a representative image 406) that represent similar semantic objects to the query medical image 402. The analytics server can use the feature embedding to map the identified regions of the support medical regions depicted in the representative image 406 to the query medical image 402, which is represented in an image 408. The analytics server can identify the labels of the locations from the support medical regions and propagate or otherwise copy the labels to the objects depicted in the corresponding locations of the query medical image to contour the query medical image. In doing so, the analytics server can create a contoured image 410 with an organ contoured.

The analytics server can train the machine learning based on the contoured image 410. For example, the analytics server can use a labeled image indicating the ground truth of the contouring of the query medical image 402 with a loss function and backpropagation techniques to adjust the weights of the machine learning model proportional to any differences between the contoured image 410 and the ground truth image. In another example, the analytics server can train the machine learning model taking the contoured image 410 as the ground truth image and feeding the contoured image 410 into the machine learning model to adjust the weights of the machine learning model such that the machine learning model is more likely to contour the query medical image 402 similar to the contoured image 410 if the machine learning model were to receive the query medical image 402 as input for contouring. The analytics server can similarly train the machine learning model in this way for any number of query medical images.

Referring again to FIG. 2, at step 208, the analytics server can receive a new image and generate a contouring attribute for the new image. For example, the analytics server can train the machine learning model using the few-shot learning technique described above until determining the machine learning model is accurate to an accuracy threshold (e.g., a predetermined threshold). Responsive to determining the machine learning model is accurate to the accuracy threshold, the analytics server can deploy the machine learning model and use the machine learning model in an inference stage to contour or generate contouring attributes (e.g., labels for pixels or voxels indicating the type of structure the respective pixels or voxels represent) for individual images of individuals.

During the inference stage, the analytics server can receive a new image of an individual. The new image can be similar to the query medical images or support medical images of the training data set in that the new image can be a cross-section view of a portion of a patient (e.g., a patient seeking or receiving treatment at a radiotherapy treatment clinic). The new image can depict multiple structures, including a first structure (e.g., a first bone or organ) and a second structure (e.g., a second bone or organ) of the patient. The analytics server can execute the trained machine learning model using the new image as input. The second structure may be an organ that is more commonly contoured, such as a lung or spinal cord, and the first structure may be an organ that is not commonly contoured, such as a pancreas or spleen. The machine learning model can determine the type of the first structure by identifying the more commonly contoured second structure and using one or more spatial relationships between the second structure and the first structure from the training data to identify the location of the first structure in the new image. The machine learning model can use the determined type of structure to contour or otherwise label the first structure when contouring the first structure. The contour outlining the first structure and/or the type of the contour can be a single contouring attribute or separate contouring attributes of the new image. The machine learning model can similarly contour any structures depicted in the new image based on spatial relationships between the structures depicted in the new image.

In some embodiments, the machine learning model can generate a confidence score for the contouring attribute for the first structure. The machine learning model can generate the confidence score for the contouring attribute based on the spatial correspondence between the first structure and the second structure in the new image. For example, the machine learning model can generate the confidence score based on a magnitude and/or direction of the distance between the first structure and the second structure as depicted in the new image compared with a magnitude and/or direction of the distance between the first structure and the second structure in one or more query medical images and/or set of support medical regions depicting the same anatomical region used to train the machine learning model. The machine learning model can compare the magnitude and/or directions of the new images with the query medical images and/or set of support medical images (e.g., an aggregation or average magnitude and/or direction in the training query medical images and/or sets of support medical images). In doing so, the machine learning model can determine a higher confidence score the closer the magnitude and/or direction of the distance between the first and second structures in the new image with the magnitude and/or direction of the first and second structure in the training query medical images and/or sets of support medical images.

In some cases, the machine learning model can generate confidence scores for the contouring attribute for individual pixels. The machine learning model can do so by determining the confidence score directly for the individual pixels and/or by determining confidence scores for groups of pixels that the machine learning model identifies or determines and of which the pixels are a part. For example, the machine learning model can determine or generate individual pixel confidence scores at a pixel level (e.g., a different confidence score for each pixel) by performing a magnitude and distance comparison for individual pixels of the new image with corresponding pixels in the corresponding training query medical images. The machine learning model can determine or generate confidence scores at a region level by performing a similar comparison for groups of pixels of a defined size (e.g., grouped to have a defined width and/or length) or grouped based on region (e.g., pixels identifying individual structures, such as the first structure and/or second structure). The machine learning model can perform a similar comparison for individual regions determined or identified by the machine learning model (e.g., using object recognition techniques or according to a defined set of rules, such as on a per group of pixels basis) of the new image compared with corresponding groups of pixels in the corresponding training query medical images. In some cases, the machine learning model can perform the comparisons based on comparisons at the pixel level and the group of pixels level in which the machine learning model generates a confidence score for the contouring attribute based on a combination, aggregation, average, median, etc., of the confidence score determined for the pixel itself and the group of pixels of which the pixel is a part.

In some cases, the machine learning model can generate the confidence score for the contouring attribute based on an uncertainty estimate of the training dataset. For example, the analytics server can match a training medical image to one or more training support medical images as described above. In doing so, the analytics server can determine a similarity score between the query medical image and each of the support medical images. The similarity score can represent an amount of uncertainty in a match between a support medical image and a query medical image. The analytics server can determine an aggregate (e.g., average or median) similarity score from the similarity scores. The analytics server can weight or adjust confidence scores that the machine learning model determines or generates for new images using the spatial correspondence between structures in the query medical image based on the aggregate similarity score of the support medical images that the analytics server determines or generates for the query medical image. For instance, the machine learning model may increase the confidence score for the contouring attribute for a higher aggregate similarity score and/or decrease the confidence score for the contouring attribute for a lower aggregate similarity score. By doing so, the machine learning model may reduce generating hallucinations or overconfident contouring attributes that may result from bad training data.

In some cases, the machine learning model can use the uncertainty estimate on a regional level. For example, the machine learning model can determine similarity scores between corresponding regions of the training query medical image and the individual support medical images matched to the training query medical image. The machine learning model can do so, for example, by identifying the regions determined to correspond to the same structure or area of an individual, such as by using object recognition techniques and/or by grouping pixels of a defined size (e.g., defined width and/or length). The machine learning model can generate embeddings of each determined region of the query medical image and the support medical images and compare embeddings of the query medical image with the support medical images determined to correspond to the same structure or area of an individual. The machine learning model can use a direct matching technique or a cosine similarity technique to determine a similarity (e.g., structure similarity score or region similarity score) between the corresponding regions and store the similarities in memory with the querying medical image and support medical images. Subsequently, when generating the confidence score for the contouring attribute for a corresponding (e.g., the same) region in the new image, the machine learning model can adjust the confidence score for the contouring attribute based on the size of the similarity for the same region in the query medical image., such as by increasing the confidence score for a similarity or decreasing the confidence score for a lower similarity. In some cases, the machine learning model can use the regional similarity in addition to the image-based similarity to adjust the confidence score for the confidence score for the region in the new region.

A benefit of using similarity on an image and/or regional level of the query medical image and the matching support medical images is it reduces the need for ensemble methods or test-time augmentation to estimate epistemic uncertainty. Instead, the uncertainty can directly stem from quantifying the dissimilarities between the query medical image and available support medical images, which can be considered when generating the confidence score for the contouring attribute and ultimately determining the contouring attribute.

The machine learning model can use the confidence score generated for the contouring attribute to determine the contouring attribute. For example, the machine learning model can determine the confidence score (e.g., the confidence score determined based on the factors described above) for the contouring attribute to a threshold (e.g., a defined threshold). Responsive to determining the confidence score exceeds the threshold, the machine learning model can output the contouring attribute. In doing so, the machine learning model can compare confidence scores for the contouring attribute to the threshold for individual pixels of the new image. The machine learning model can output the contouring attribute as a label for each of the pixels indicating whether it depicts an outline of a structure and/or a type of the structure based on the confidence score for the outline and/or the type of the structure exceeding the threshold. In doing so, the machine learning model can generate and/or output labels for one or more structures depicted in the new image. The contouring attribute of the first structure can be or include one or more labels of pixels indicating the outline of the first structure and/or a type of the first structure.

At step 210, the analytics server may transmit the contouring attribute of the first structure to a radiotherapy computer model. The radiotherapy computer model can be or include a machine learning model or an optimization model configured to generate radiotherapy treatment plans for patients. The radiotherapy treatment plans can include treatment attributes, such as different treatment modalities, field geometry settings for external beam radiotherapy, side effect predictions, organ and/or tumor segmentation, machine therapy attributes, dosage administration attributes (e.g., dosage amount), treatment frequency, treatment timing, monitored units, etc.

The radiotherapy computer model may be stored locally by the analytics server or stored by a remote computing device or system. In cases in which the radiotherapy computer model is stored locally, the analytics server can transmit the contouring attribute of the first structure to the radiotherapy computer model by retrieving the radiotherapy computer model from memory or storage and inputting the contouring attribute into the radiotherapy computer model, in some cases with any other patient data the analytics server has stored for the patient and/or contouring attributes the analytics server generates (e.g., using the machine learning model) from the new image and any other image of the patient. In cases in which the radiotherapy computer model is stored by a remote computing device or system, the analytics server can transmit (e.g., over a wired or wireless communications network) the contouring attribute with any other data regarding the patient as described above to the remote computing system or device. The remote computing system or device can receive the data and input the data into the radiotherapy computer model.

In some cases, the analytics server can use the few-shot learning technique described herein during inference. For example, the analytics server can receive, retrieve, or obtain the new image containing the first structure and the second structure with a request to segment the new image. Responsive to doing so, the analytics server can retrieve an inference set of medical images from a database. The inference set of images can be the same as or similar to the contoured support medical images, as described herein. The analytics server can retrieve and/or select the inference set of medical images based on similarities between the new medical image and the inference set of medical images. For example, the analytics server can generate an embedding from the new image and compare the embedding with embeddings generated (e.g., in the same vector space) of the inference set of images. Based on the comparison, the analytics server can identify and/or select the inference set of medical images as a defined number of inference medical images with the highest similarity scores and/or with similarity scores that satisfy a threshold (e.g., a defined threshold). The selected inference set of medical images can depict the first structure and the second structure.

The analytics server can use the retrieved inference set of medical images to contour the new image. To do so, the analytics server can generate an embedding representing or corresponding to the spatial correspondence between the structures depicted in the new image and the structure depicted in the retrieved inference set of medical images. The embedding can indicate a spatial relationship between the first and second structures in each image of the inference set of images. The embedding can additionally or instead indicate a spatial relationship between the second structure in the inference set of images and the new image. The analytics server can input the embedding with the new image and the selected set of inference images into the machine learning model. Based on the input and responsive to execution of the machine learning model, the machine learning model can propagate the contouring labels of the first structure from the inference set of images to the new image. The machine learning model can do so by identifying the second structure in the new image and using the spatial correspondence between the second structure in the new image and the second structure in the inference set of images. The machine learning model can use the spatial correspondence between the first structure and the second structure in the inference set of images and the spatial correspondence between the first structure and the second structure in the new image to identify the first structure at a location in the new image. The machine learning model can generate a contouring attribute labeling the outline of the first structure in the new image based on the identification. The analytics server can transmit the contouring attribute of the first structure to the radiotherapy computer model configured to generate a radiotherapy treatment plan for treatment of the first structure and/or control a radiotherapy machine for the treatment according to the radiotherapy treatment plan.

At step 212, the radiotherapy computer model can generate the radiotherapy treatment plan for the patient depicted by the new image using the contouring attribute. For example, if the analytics server stores the radiotherapy treatment plan, the analytics server can execute the radiotherapy model using the contouring attribute and any other patient data for the patient to generate the radiotherapy treatment plan. If a remote computing system or device stores the radiotherapy treatment plan, the remote computing system or device can execute the radiotherapy model to generate the radiotherapy treatment plan for the patient. In some cases, the remote computing system or device can transmit the generated radiotherapy treatment plan back to the analytics server.

The analytics server can control a radiotherapy machine according to the radiotherapy treatment plan (e.g., according to dosage and positioning attributes of the radiotherapy treatment plan). For example, the analytics server can configure (e.g., automatically configure) the radiotherapy machine according to the attributes identified in the radiotherapy treatment plan. The analytics server can activate the radiotherapy machine to treat the patient for which the analytics server generated the radiotherapy treatment plan based on the new image received of the patient.

In some cases, the analytics server can present, the new medical image on a display of a computing device, such as a monitor or display of the radiotherapy machine or another machine. The analytics server can present the medical image showing a contour outlining the first structure generated based on the contouring attribute of the first structure (e.g., the labels outlining the first structure and/or identifying a type of the first structure). A user, such as a medical professional or a patient can view the contoured medical image to determine steps for treatment and/or determine an overall health of the first structure.

In some cases, implementing the systems and methods described herein can facilitate auto-contouring according to rules that are specific to individual providers (e.g., medical professionals) and/or radiotherapy clinics. For example, the analytics server can contour the first structure or generate or predict the contouring attribute for the first structure of the method 200 based on a clinic-specific rule or a second rule specific to a medical professional. This can be accomplished through the contouring of the support medical images used during the training phase. For instance, radiation therapy oncology group (RTOG) guidelines for femoral head contouring specify inclusion of the head, neck and shaft down to the ischial tuberosity. However, some providers follow alternate protocols outlining only the head. Rather than re-developing an entirely new model, a user can include one or more support medical images that include contours reflecting the user's preferences. The support medical images can be used in the training phase to train the machine learning model such that the machine learning model learns or otherwise is weighted to propagate the user's preference to new cases without exhaustive manual edits. By annotating just a few examples (potentially only one), the user can customize the segmentation without requiring model re-training or annotating each new case from scratch.

In a non-limiting example, a computer implementing the systems and method described herein can generate a training dataset including a set of medical images, such as CT scans, that depict various anatomical regions of individuals'bodies. To do so, the computer can identify query medical images that are not contoured and support medical images that are contoured (e.g., contoured and labeled to indicate structures within the body, such as tumors and/or organs-at-risk (OARs). The computer can match individual query medical images to one or more support medical images based on the support medical images depicting the same anatomical region as the query medical image and/or having a similarity score with the query medical image that satisfies one or more criteria (e.g., exceeds or satisfies a threshold and/or is the highest of a group of support medical images depicting the same anatomical region). The training dataset can include any number of query medical images matched to support medical images.

For each query medical image in the training dataset, the computer can generate (e.g., using an embedding model, such as an encoder or another type of machine learning model) a feature embedding for the query and support medical images (e.g., only the support medical images). The embedding can be or include a numerical vector representing the spatial correspondences between the structures within the query medical image and/or those within each support medical image and/or the spatial correspondence between the structures depicted in the query medical image and/or the structures depicted in the support medical images. In some cases, the computer can generate separate embeddings for the query medical image and each support medical image matched to the query medical image indicating the spatial relationships between the structures depicted within the respective medical images. In such cases, the computer can generate a separate embedding indicating the spatial relationship between the same types of structures depicted in the query medical image and the respective support medical images. The computer can similarly generate such embeddings for any number of query medical images in the training dataset. In doing so, the computer can identify and encode the spatial relationships between structures, causing the embedding or embeddings to reflect the anatomical similarities and differences between medical images.

The computer can use the training dataset and corresponding feature embeddings to train a machine learning model. The computer can iteratively input the individual query medical images, support medical images matched to the query medical images, and the one or more embeddings generated for the query medical images and support medical images into the machine learning model. Based on the inputs, the machine learning model can propagate the contouring in the support medical images to the query medical images using the embeddings of the query medical image and/or the support medical images. The computer can train the machine learning model based on the newly contoured query medical images by using the newly-contoured query medical images as the ground truth to adjust the weights and/or parameters of the machine learning model and/or by comparing the contouring of the query medical images to a ground truth medical images and using a loss function with backpropagation techniques to train the machine learning model. In doing so, the computer can train the machine learning model to use spatial relationships between structures to according to the embedding or embeddings generated for the query medical image and/or support medical images. Over several iterations, the machine learning model can refine its ability to predict contouring attributes (e.g., labels indicating an outline or type of a structure depicted in an image) for new medical images based on the spatial correspondence or relationships between structures depicted within individual images.

Once the machine learning model is trained (e.g., trained to an accuracy threshold), the analytics server can use the machine learning model to contour new images. For instance, the analytics server can receive a new CT scan as input. The analytics server can execute the trained machine learning model based on the new CT scan to contour or generate one or more contouring attributes for the structures depicted in the new CT scan. In one example, if the new image depicts a lung, the machine learning model can use its trained weights and/or parameters to identify the spatial correspondence between the lung and other structures depicted in the CT scan to identify the other structures and/or types of the other structures that are depicted in the CT scan. The machine learning model can predict the contouring attributes, such as the boundaries and/or the types, of the other structures based on the identification using the spatial correspondence with the lung.

The analytics server can transmit the contouring attributes predicted for the new image to a radiotherapy computer model. The radiotherapy computer model can be specifically configured to generate radiotherapy treatment plans including different treatment attributes that can be used by a radiotherapy machine to treat a patient. The radiotherapy computer model can use the contouring attribute to generate such treatment attributes. For example, the radiotherapy computer model can use the contouring attribute to generate treatment attributes for a position of a radiotherapy treatment machine to optimize the radiation dose distribution, such as to increase the dose of radiation that the radiotherapy machine provides to a tumor while sparing healthy tissues and/or organs-at-risk. The analytics server can control the radiotherapy machine according to the generated radiotherapy treatment plan to provide treatment to the patient.

Advantageously, by implementing the systems and methods described herein, the analytics server can use the few-shot learning technique described herein to train the machine learning model to contour structures depicted in medical images in an efficient and more accurate manner. Because the training involves spatial correspondence between structures of multiple images, the training may facilitate the machine learning model being able to more accurately contour structures of images for which there is little training data available than other training methods.

The framework can enable contouring novel structures not currently supported, such as uncommon OARs or tumor volumes like GTV, CTV, PTV. As with contour customization, providing a few annotated examples as support medical images can allow automatic delineation of the new structure subsequently. More support medical images may be required for highly variable targets, like tumors, versus intrinsic anatomical variation. Accurate support medical image selection is important, and can be approached as an active learning and similarity-based retrieval problem. Potential strategies include (1) batch-based and (2) iterative. In batch-based, a set of queries are collected upfront, then the most similar support medical images are retrieved for each query from the database. In iterative, it begins with a limited set of support medical images to progressively improve performance through human-in-the-loop correction. After correcting initial suboptimal contours, the verified images augment the support pool for upcoming queries. Over successive iterations, segmentation of the novel structure is expected to stabilize. Supplying a few initial examples facilitates extension to new structures. Smart support retrieval and incremental update approaches can help drive performance as the number of support medical images increases.

Another advantage of implementing the systems and methods described herein is efficient annotation. For development of new structures, the proposed method can be used to boost the efficiency of annotations efficiently. For example, an iterative human-in-the-loop schema can be deployed to develop any structure contouring, and not many support medical images may need to be annotated. In addition, a project can be to annotate all possible anatomical structures with fine granularities of a whole body image, and use that as the genesis support, spawn into more support medical images that require only minor edits for correction. Once enough support medical images are annotated and curated, they can either be used for training a supervised model for all the anatomical structures, or as a fixed set of high quality support for the few-shot learning segmentation described herein. The proposed method can be combined with specialist model already supported. The combination can be (1) brute force: use dedicated model for organs that are supported, use approach described herein when there is only support available, (2) integrated: all segmentation route to the approach described herein, for organs with dedicated models, apply dedicated models on cases most similar to the query, and consider these selected cases as support medical images, the performances of the integrated combination needs to be evaluated.

Implementing the systems and methods described herein can also enhance the efficiency of annotating training data for new structures. Similar to the iterative strategy mentioned above, a human-in-the-loop workflow can reduce total annotation efforts. Sparsely labeling an initial genesis support set can propagate to expanded coverage through minor correction. Once curated support medical images meet sufficiency criteria, they can either train supervised specialist models or serve directly for few-shot segmentation.

Additionally, implementing the systems and methods described herein can involve whole-body automation. The initiative can delineate all anatomical structures with comprehensive coverage in a single image, propagating these initial support medical images to other images (e.g., query medical images) with minimal incremental labeling until convergence.

The auto-contouring solution framework described herein can be compatible with existing specialist models. For instance, the integration strategy can be brute force or unified. Brute force uses existing specialist models to delineate already supported structures, few-shot models to handle novel structures with support medical images. Unified uses leverage specialist models to provide support medical images for few-shot learning for supported structures, and thus all contouring results will be generated from the described approach.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
generating, by at least one processor, a training dataset comprising a set of medical images comprising:
at least one query medical image depicting one or more structures within an anatomical region, and
a plurality of support medical images depicting the one or more structures within the anatomical region, wherein the one or more structures are contoured and labeled;
generating, by the at least one processor, a feature embedding for the at least one query medical image and the plurality of the support medical images, the feature embedding corresponding to a spatial correspondence between the one or more structures within the query medical image and the one or more structures within each support medical image;
training, by the at least one processor, a machine learning model using the training dataset and the feature embedding, such that the machine learning model is configured to receive a new medical image and predict a contouring attribute for a first structure within the new medical image in accordance with a spatial correspondence to a second structure within the new medical image;
and transmitting, by the at least one processor, the contouring attribute of the first structure to a radiotherapy computer model configured to generate a radiotherapy treatment plan for treatment of the first structure.

2. The method of claim 1, wherein the machine learning model is configured to predict the contouring attribute for the first structure within the new medical image based on a clinic-specific rule or a second rule specific to a medical professional.

3. The method of claim 1, further comprising:
storing, by the at least one processor, a set of two-dimensional (2D) support medical images of one or more individuals in memory; and
selecting, by the at least one processor, a subset from the set of 2D support medical images that correspond to the anatomical region in the query medical image.

4. The method of claim 3, wherein selecting the subset of the set of 2D support medical images comprises:
selecting, by the at least one processor, the subset of the set of 2D support medical images based on similarity scores between the at least one query medical image and the subset of the set of 2D support medical images.

5. The method of claim 1, further comprising:
generating, by the at least one processor, a confidence score for the contouring attribute based on the spatial correspondence between the first structure and the second structure in the new medical image.

6. The method of claim 5, wherein generating the confidence score comprises:
generating, by the at least one processor, the confidence score based on an aggregation of pixel confidence scores for the contouring attribute of the new medical image.

7. The method of claim 1, further comprising:
presenting, by the at least one processor, for display, the new medical image comprising a contour of the first structure.

8. The method of claim 1, wherein one or more of the plurality of support medical images depict a spatial relationship between the second structure and first structure, the plurality of support medical images each depicting a contour of the second structure.

9. The method of claim 8, wherein the machine learning model is configured to predict the contouring attribute for the first structure within the new medical image based on the spatial relationship between the second structure and the first structure in the one or more of the plurality of support medical images.

10. The method of claim 1, further comprising:
retrieving, by the at least one processor, an inference set of medical images from a database based on similarities between the new medical image and the inference set of medical images,
wherein the machine learning model is configured to generate the contouring attribute for the first structure based on the retrieved inference set of medical images.

11. The method of claim 10, wherein one or more of the inference set of medical images comprising a contouring of the first structure; and
wherein the machine learning model is configured to generate the contouring attribute for the first structure in the new medical image based on a spatial correspondence between the first structure and the contouring of the first structure in the one or more of the inference set of medical images.

12. A system comprising:
a non-transitory computer-readable medium storing instructions that when executed cause at least one processor to:
generate a training dataset comprising a set of medical images comprising:
at least one query medical image depicting one or more structures within an anatomical region, and
a plurality of support medical images depicting the one or more structures within the anatomical region, wherein the one or more structures are contoured and labeled;
generate a feature embedding for the at least one query medical image and the plurality of the support medical images, the feature embedding corresponding to a spatial correspondence between the one or more structures within the query medical image and the one or more structures within each support medical image;
train a machine learning model using the training dataset and the feature embedding, such that the machine learning model is configured to receive a new medical image and predict a contouring attribute for a first structure within the new medical image in accordance with a spatial correspondence to a second structure within the new medical image; and
transmit the contouring attribute of the first structure to a radiotherapy computer model configured to generate a radiotherapy treatment plan for treatment of the first structure.

13. The system of claim 12, wherein the machine learning model is configured to predict the contouring attribute for the first structure within the new medical image based on a clinic-specific rule or a second rule specific to a medical professional.

14. The system of claim 12, wherein the instructions further cause the at least one processor to:
store a set of two-dimensional (2D) support medical images of one or more individuals in memory; and
select a subset of the set of 2D images from the set of 2D support medical images that correspond to the anatomical region in the query medical image.

15. The system of claim 14, wherein the instructions cause the at least one processor to select the subset of the set of 2D support medical images by:
selecting the subset of the set of 2D support medical images based on similarity scores between the query medical image and the subset of the set of 2D support medical images.

16. The system of claim 12, wherein the instructions further cause the at least one processor to:
generate a confidence score for the contouring attribute based on the spatial correspondence between the first structure and the second structure in the new medical image.

17. The system of claim 16, wherein instructions cause the at least one processor to generate the confidence score by:
generating the confidence score based on an aggregation of pixel confidence scores for the contouring attribute of the new medical image.

18. The system of claim 12, wherein the instructions further cause the at least one processor to:
present, for display, the new medical image comprising a contour of the first structure.

19. The system of claim 12, wherein one or more of the plurality of support medical images depict a spatial relationship between the second structure and first structure, the plurality of support medical images each depicting a contour of the second structure.

20. The system of claim 19, wherein the machine learning model is configured to predict the contouring attribute for the first structure within the new medical image based on the spatial relationship between the second structure and the first structure in the one or more of the plurality of support medical images.
